SEQUENCE LISTING (9 sequences)

SEQ ID NO: 1 - protein, 12 aa, Plasmodium falciparum
EVMEISFDNDYI

SEQ ID NO: 2 - protein, 15 aa, Plasmodium falciparum
EIIEIVFDENEEKYF

SEQ ID NO: 3 - protein, 13 aa, Plasmodium falciparum
NEVENENEDFNDI

SEQ ID NO: 4 - protein, 15 aa, Plasmodium falciparum
ETLVPVDDDSDMWME

SEQ ID NO: 5 - protein, 12 aa, Plasmodium falciparum
EQFRLPEENEWN

SEQ ID NO: 6 - protein, 14 aa, Plasmodium falciparum
DNKAMDEEEFWALE

SEQ ID NO: 7 - protein, 14 aa, Plasmodium falciparum
EDFEVVDADDPMWN

SEQ ID NO: 8 - protein, 12 aa, Plasmodium falciparum
QNIEIMNDTQWK

SEQ ID NO: 9 - protein, 12 aa, Plasmodium falciparum
HTTPVLMEKPYY

(19) United States
(12) Patent Application Publication
(10) Pub. No.: US 2018/0087086 A1
(43) Pub. Date: Mar. 29, 2018

(54) CELL EGRESS AND INVASION INHIBITORS AND THEIR USE AS ANTIPARASITICAL AGENTS

(71) Applicant: THE JOHNS HOPKINS UNIVERSITY, Baltimore, MD (US)

(72) Inventors: Jürgen Bosch, Pikesville, MD (US); Lauren E. Boucher, Baltimore, MD (US)

(21) Appl. No.: 15/567,123

(22) PCT Filed: Apr. 15, 2016
(86) PCT No.: PCT/US2016/027665

§ 371 Date: Oct. 17, 2017

Related U.S. Application Data
(60) Provisional application No. 62/149,240, filed on Apr. 17, 2015.

(51) Int. Cl. C12Q 1/18; A61K 45/06; A61K 31/426; A61K 31/498; A61K 31/4184; A61K 31/4985; A61K 31/454

(52) U.S. Cl. CPC C12Q 1/18; A61K 45/06; A61K 31/426; A61K 31/454; A61K 31/4184; A61K 31/4985; A61K 31/498

(57) ABSTRACT

The present invention provides cell egress and invasion inhibitors and their use as antiparasitical agents.

### REFERENCE TO RELATED APPLICATIONS

This application claims the benefit of U.S. Provisional Patent Application No. 62/149,240, filed on Apr. 17, 2015, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Apr. 11, 2016, is named P13219-02_ST25.txt and is 2,010 bytes in size.

### BACKGROUND OF THE INVENTION

Plasmodium parasites cause malaria, which is one of the most devastating infectious diseases. The emergence of drug resistance by Plasmodium falciparum necessitates the development of antimalarial compounds to stay one step ahead of the rapidly-evolving parasite. Approximately ⅓ of the world's population is at risk of infection, with approximately 500 million infections, and 0.6-1 million deaths annually. A key component of the invasion machinery is a tetrameric complex of Aldolase that connects the motor to the cytoplasmic domain of a transmembrane adhesive protein, TRAP (thrombospondin related anonymous protein). As such, there is a pressing need to identify compounds that disrupt the TRAP-Aldolase interaction, and thereby preventing parasite invasion of a host cell.

Malaria is usually confirmed by the microscopic examination of blood films or by antigen-based rapid diagnostic tests (RDT). Microscopy is the most commonly used method to detect the malarial parasite. Despite its widespread usage, diagnosis by microscopy suffers from two main drawbacks: many settings (especially rural) are not equipped to perform the test, and the accuracy of the results depends on both the skill of the person examining the blood film and the levels of the parasite in the blood. The sensitivity of blood films ranges from 75-90% in optimum conditions, to as low as 50%. Commercially available RDTs are often more accurate than blood films at predicting the presence of malaria parasites, but they are widely variable in diagnostic sensitivity and specificity depending on manufacturer, and are unable to tell how many parasites are present.

Malaria is classified into either “severe” or “uncomplicated” by the World Health Organization (WHO). It is deemed severe when any of the following criteria are present, otherwise it is considered uncomplicated: Decreased consciousness; Significant weakness such that the person is unable to walk; Inability to feed; Two or more convulsions; Low blood pressure (less than 70 mmHg in adults and 50 mmHg in children); Breathing problems; Circulatory shock; Kidney failure or hemoglobin in the urine; Bleeding problems, or hemoglobin less than 50 g/L (5 g/dL); Pulmonary oedema; Blood glucose less than 2.2 mmol/L (40 mg/dL); Acidosis or lactate levels of greater than 5 mmol/L; A parasite level in the blood of greater than 100,000 per microlitre (μL) in low-intensity transmission areas, or 250,000 per 4 in high-intensity transmission areas. Cerebral malaria is defined as a severe P. falciparum-malaria presenting with neurological symptoms, including coma (with a Glasgow coma scale less than 11, or a Blantyre coma scale greater than 3), or with a coma that lasts longer than 30 minutes after a seizure.

### SUMMARY OF THE INVENTION

The invention is based, in part, upon the surprising discovery that compounds that modulate the interaction between Plasmodium aldolase and a transmembrane adhesive protein are useful in treating or preventing malaria.

In accordance with an embodiment, the present invention provides the use of an agent that modulates the interaction between Plasmodium aldolase and a transmembrane adhesive protein for use in treating a Plasmodium infection in a subject.

In accordance with another embodiment, the present invention provides the use of an agent that modulates the interaction between Plasmodium aldolase and a transmembrane adhesive protein for use in inducing cell death in a Plasmodium spp.

In accordance with a further embodiment, the present invention provides a method of identifying a candidate compound that modulates the activity of Plasmodium aldolase comprising contacting Plasmodium aldolase with a candidate compound and determining whether said candidate compound modulates the interaction between Plasmodium aldolase and a transmembrane adhesive protein.

### BRIEF DESCRIPTION OF THE DRAWINGS

FIG. 1 is a schematic of the invasion machinery, also known as the glideosome. The machinery consists of an actomyosin motor that is attached to the parasite via the myosin tail interacting protein (MTIP) linking myosin to the inner membrane complex (IMC) and associated GAP (guanosine-5′-triphosphate-activating) proteins. To transmit the locomotive force of the actomyosin motor to the extracellular attachments, the glycolytic enzyme aldolase assumes a structural bridging role, binding to both aldolase and the carious C-terminal cytosolic adhesion tails, including those of the trap family.

FIG. 2A-FIG. 2D is a series of illustrations and a table that show the aldolase-TRAP interaction. FIG. 2A is a schematic of the tetrameric complex of aldolase. FIG. 2B illustrates the conservation of the P. falciparum Aldolase active site with human, mouse, and rabbit aldolase indicating a highly conserved pocket. The left shows the apo structure, and the right shows the TRAP peptide bound structure. FIG. 2C is a table of the TRAP protein family members and the domain schematics and stage information. FIG. 2D is a sequence alignment of various cytoplasmic tails of extracellular adhesions known to bind aldolase.

FIG. 3 shows the Aldolase-TRAP specific interaction measurement via surface plasmon resonance. The dissociation constant for PfAldolase and PfTRAP, MTRAP and PvTRAP was measured by determining the binding response at various concentrations of aldolase passed over a chip with bound, biotinylated peptides. Dissociation constants were in the mid-nanomolar range and matched with values previously reported in the literature.

FIG. 4A-FIG. 4C is a series of graphs that show the screening of the MMV library for compounds that promote the TRAP-Aldolase interaction. Graphs show response in the presence of compound normalized to the control (DMSO) (dimethyl sulfoxide) which was set to zero. Positive responses indicated increased interaction between TRAP (FIG. 4A) or MTRAP, and aldolase and negative response indicated a disruption of the interaction (FIG. 4B). FIG. 4C shows selected binding curves from the screen. The control curve is shown in black. Examples of slow dissociation with normal binding (blue), increased binding with normal dissociation (green), increased binding with fast dissociation (pink) and disruption of the interaction (purple) are shown.

FIG. 5A and FIG. 5B is a series of line graphs and a dot plot that show the increased binding interaction of (M)TRAP with aldolase in the presence of compound as a function of concentration. FIG. 5A (Top) TRAP and FIG. 5B (Bottom) MTRAP show the interaction with aldolase in the presence of compounds 4 and 18 as a function of compound concentration as measured via SPR.

FIG. 6A and FIG. 6B are a series of photomicrographs that show inhibition of gliding motility in the presence of a compound. Images of parasite CSP trails shed during gilding on antibody coated slides in the presence of decreasing concentrations of compound. Compound 4 (FIG. 6A) and compound 18 (FIG. 6B) effects on gliding are shown in comparison to DMSO control (right panels).

FIG. 7 is a series of bar graphs that show quantification of decreased gliding of parasites in presence of compound. Parasites were binned based on the number of trails with which they were associated. Five compounds from the initial MMV SPR screen were tested and are shown alongside a DMSO control.

FIG. 8 is a dot plot that shows analysis of phenotypic variation in trail radius of gliding sporozoites in the presence of compound. The trail radius was measured using ImageJ and the statistical analyses to determine standard error mean and t-test were performed using GraphPad.

FIG. 9 is a series of photomicrographs that show representative fields of views for sporozoites in the presence of each compound (3, 4, 17, 18 and 23) ranging from 250 μM to 0 μM in twofold dilution series.

FIG. 10 is a series of photomicrographs that show representative views of sporozoite in the presence of compounds.

FIG. 11A is a dot plot that shows live imaging gliding radius of sporozoites in the presence of compounds 4 and 18. FIG. 11B is a dot plot that shows sporozoite circular gliding velocity in the presence of compounds 4 and 18. FIG. 11C is a dot plot that shows time spent gliding in the presence of compounds 4 and 18. FIG. 11D is a dot plot that shows the fraction of time spent paused during active gliding in the presence of compounds 4 and 18. FIG. 11E is a bar chart that shows the ratio of waving versus circular gliding in the presence of compounds 4 and 18. FIG. 11F shows a cartoon depicting waving and gliding patterns of movements in sporozoite motion.

FIG. 12A-FIG. 12C is a series of bar charts that show results of sporozoite viability assays. FIG. 12A shows sporozoite viability in the propidium iodine exclusion assay viability test. The total number of sporozoites was determined from phase images and the number of non-viable sporozoites from the count of red fluorescing sporozoites. FIG. 12B shows sporozoite viability using a 2 dye system of ethidium and calcein-AM to measure both membrane permeability, a marker of death, and enzyme activity, a marker of viability.

FIG. 12C shows sporozoite viability using the LIVE/DEAD fixable violet assay.

FIG. 13A is a bar graph that shows gliding assay results with compound washouts, indicating partial recovery of motility when the compound is removed. FIG. 13 B is a series of photomicrographs that show representative images from slides of gliding sporozoites. DMSO treated sporozoites (a, upper left) with circular gliding pattern versus (b, upper right) pyrazole-urea compound treated sporozoites with a unique wavy line gliding pattern. Below are images of a compound 4 washout slide at 7.8 μM showing a similar wavy gliding patterns in addition to circular gliding (c, d).

FIG. 14 A is a bar graph that shows HepG2 invasion assay results in the presence and absence of compounds. FIG. 14B is a bar graph that shows HepG2 viability using propidium iodine exclusion assays in the absence and presence of compounds. FIG. 14C is a series of photomicrographs that show LIVE/DEAD assay of compound 4 at 62.5 μM compared to DMSO control. FIG. 14 D is a photograph of a mouse tail used for testing of compound 4 and compound 18 as a topical agent. Compounds were applied onto the mouse tail solubilized in 100% DMSO at a concentration of 2.5 mM (˜100 fold of EC₉₀). Infected Anopheles stephensi mosquitoes carrying the P. berghei rodent malaria parasite were allowed to feed on the treated tails.

FIG. 15 is a bar chart that shows that dose-dependent killing of P. falciparum sporozoites by the compounds described herein. Compound 18 EC₉₀˜30 μM and compound 4 EC₉₀˜125 μM. Observed phenotype at lower concentration pinpoints towards an on-target effect of the compounds.

FIGS. 16A-16D depict an aldolase enzyme activity assay. (A) Cartoon and surface representation of aldolase with TRAPpep bound (magenta, sticks) (PDB entry 2pc4), overlapping the F16BP (green, sticks) substrate binding site (PDB entry 4ald), via PyMOL. (B) Coupled enzyme assay to measure aldolase activity via NADH consumption as measured at 340 nm. (C) Concentration-dependent effect of compounds on PfAldolase inhibition in the presence of PbTRAP peptide. Activity is normalized to the PbTRAP control, which reduces Vₘₐₓ to 50% of enzyme without PbTRAP. (D) PfAldolase activity in the presence of compounds to determine direct inhibition of enzyme by the compounds. Activity is normalized to the PfAldolase control measured Vₘₐₓ. Standard error means plotted for activity assays and standard deviation for SPR data.

### DETAILED DESCRIPTION OF THE INVENTION

Plasmodium parasites cause malaria, which is one of the most devastating infectious diseases. The emergence of drug resistance by P. falciparum necessitates the development of novel antimalarial compounds to stay one step ahead of the rapidly evolving parasites. Parasites have developed resistance to all known antimalarial compounds. Artemisin resistance is increasing in Cambodia. Approximately ⅓ of the world's population is at risk of infection, with approximately 500 million infections every year and 0.6-1 million deaths annually. The vast majority cases (˜90%) are children under the age of 5. The current malaria vaccine, RTS,S (Repeat region of Plasmodium falciparum circumsporozoite protein (CSP) T-cell epitopes of CSP hepatitis B surface antigen (HBsAg), spontaneously assemble) fails to protect young children (only 30% protection) due to their immature immune system. Cerebral malaria (CM) is the most severe neurological complication of infection with P. falciparum and has a 50% mortality rate. The only treatment is high dosages of chloroquine.

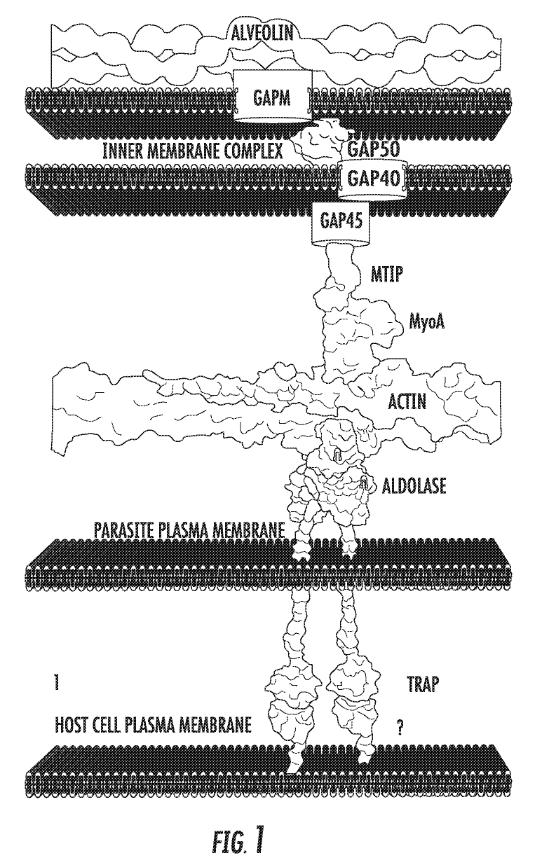

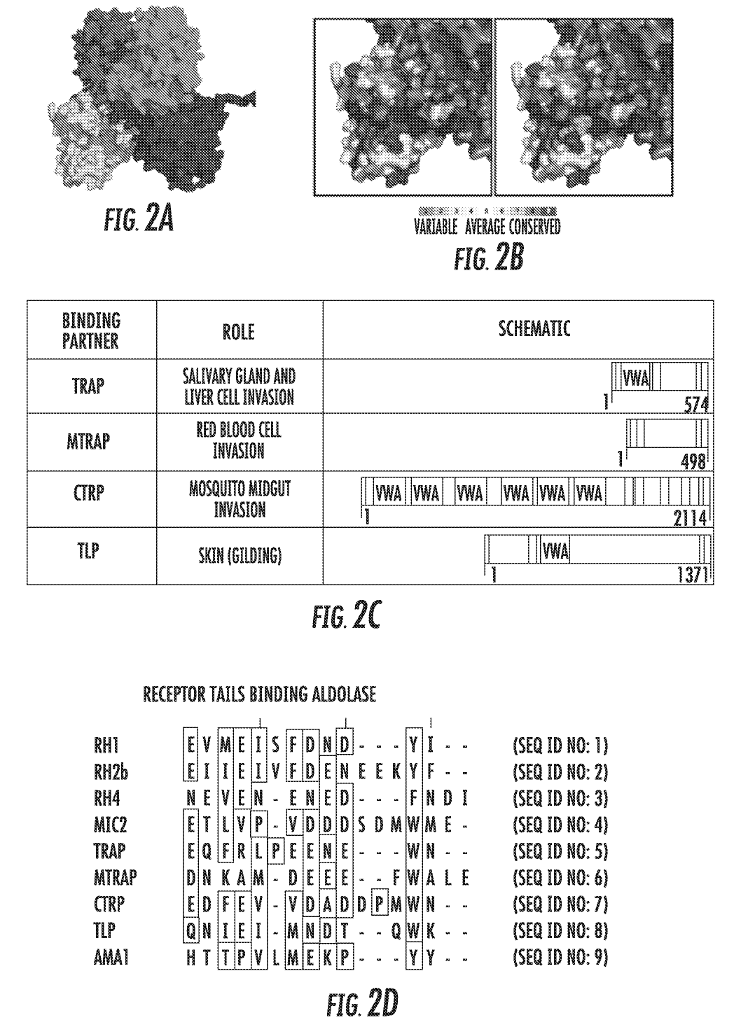

The life cycle of the Plasmodium parasite is very complex and involves various cellular interactions in the mosquito vector as well as in the human host. Sporozoites are injected during a blood meal into the skin by an infected female Anopheles mosquito (Bosch J et al, J. Struct. Biol. 2012 April 178(1): 61-73, incorporated herein by reference). After invading hepatocytes, numerous merozoites merge a few days later into the blood stream, which in their turn invade erythrocytes. After one to three days, depending on species, 10-20 new merozoites egress from erythrocytes. Throughout the life cycle of the parasite, various cell barriers need to be traversed to ensure survival and progeny of the parasite. A specialized multi-protein complex, which fulfills the function of “substrate gliding” and invasion of host cells is highly conserved throughout the phylum apicomplexa. This invasion machinery of the parasite, also called the “glideosome”, is located between the parasite plasma membrane (PPM) and the microtubule-supported inner membrane complex. The invasion machinery includes an adhesion protein (TRAP, MTRAP or CTRP, depending on the life stage of the parasite) linked via aldolase to short actin filaments. These filaments are part of the actin-myosin motor including the Myosin A-tail interacting protein (MTIP) that connects to the GAP45-GAP50 complex Compounds that can disrupt the parasite's invasion and egress pathways to prevent proliferation were identified. The parasite employs an actomyosin motor as part of a larger invasion machinery complex known as the glideosome to invade target cells (FIG. 1). A key component of the invasion machinery is a tetrameric complex of Aldolase (FIG. 2A) that connects the motor to the cytoplasmic domain of a transmembrane adhesive protein, TRAP (thrombospondin related anonymous protein) (Bosch J. et al PNAS 104; 2007; 7015, incorporated herein by reference). The conservation of the P. falciparum Aldolase active site between human, mouse, and rabbit aldolase indicates a highly conserved pocket (FIG. 2B). During invasion, aldolase serves in the role of a structural bridging protein, as opposed to its normal enzymatic role in the glycolysis pathway (Bosh J et al. Acta Cryst F; 2014 70(1186), incorporated herein by reference). Aldolase interacts with the C-terminal tail of the TRAP proteins, which are specific to different stages of invasion, such as TRAP for sporozoite invasion of liver cells, MTRAP for merozoite invasion of red blood cells, and CTRP for ookinete invasion of the mosquito midgut (FIG. 2C). A sequence alignment of cytoplasmic tails of extracellular adhesions known to bind aldolase is shown in FIG. 2D.

The Medicines for Malaria Ventures (MMV) box (Spangenberg 2013 PLoS One doi: 10.1371/journal.pone.0062906) is an open access malaria box which contains 400 diverse compounds with antimalarial activity. MMV has been screened to identify compounds that affect the TRAP-Aldolase interaction. Due to the high conservation of the Aldolase active site where TRAP tails bind, prior to the invention described herein, it was difficult to identify drugs that disrupt the complex without interfering with the host's Aldolase. As described herein, as invasion progresses, the TRAP-Aldolase complex must periodically dissociate for invasion to continue.

Targeting vital pathways and vital protein-protein interaction interfaces for inhibition is a viable approach for treatment (Drug Discov. Today, 2016 March; 21(3):491-8, incorporated herein by reference). Protein interaction interfaces have developed over millions of years, are optimized for recognition, and mutations in these areas will compromise function and are therefore undesirable.

Components of the invasion machinery of various host cells (including mosquito midgut, human liver cells and human red blood cells), are necessary for successful egress and continuation of the lifecycle of the parasite. Host cell remodeling through the parasite is a crucial pathway.

In accordance with an embodiment, the present invention provides the use of an agent that modulates the interaction between Plasmodium aldolase and a transmembrane adhesive protein for use in treating a Plasmodium infection in a subject.

In some embodiments, the agent(s) that modulates the interaction between Plasmodium aldolase and a transmembrane adhesive protein comprise small molecule compounds disclosed herein.

The compounds identified herein selectively inhibit the Plasmodium parasite Aldolase-TRAP complex and Aldolase-MTRAP complex. As described herein, inferring from sequence identity between TRAP and CTRP, the Aldolase-CTRP complex is inhibited during the mosquito stage. For example, as described in detail below, 2-(N,3-N-bis(4-bromophenyl)quinoxaline-2,3-diamine) (Compound 4) exhibits cytocidal activity at a 60 μM concentration and may be used as a topical agent for prophylaxis.

Specifically, methods of treating or preventing malaria in a subject are carried out by administering to the subject an agent that modulates the interaction between Plasmodium aldolase and a transmembrane adhesive protein. The subject is preferably a mammal in need of such treatment, e.g., a subject that has been diagnosed with malaria or a predisposition thereto. Malaria is typically diagnosed by the microscopic examination of blood using blood films or with antigen-based rapid diagnostic tests or with PCR-based assays. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Also provided are methods of inducing cell death/reducing viability in a Plasmodium spp. comprising contacting a Plasmodium spp. with an agent that modulates the interaction between Plasmodium aldolase and a transmembrane adhesive protein. Methods of inhibiting/killing Plasmodium spp. sporozoites are carried out by contacting a Plasmodium spp. sporozoite with an agent that modulates the interaction between Plasmodium aldolase and a transmembrane adhesive protein. Methods of preventing proliferation of Plasmodium spp. sporozoites are also carried out by contacting a Plasmodium spp. sporozoite with an agent that modulates the interaction between Plasmodium aldolase and a transmembrane adhesive protein.

In accordance with another embodiment, the present invention provides the use of an agent that modulates the interaction between Plasmodium aldolase and a transmembrane adhesive protein for use in inducing cell death in a Plasmodium spp.

It will be understood by those of ordinary skill in the art that the aldolase enzyme targeted in Plasmodium can also be targeted in a number of other parasite species. For example, these compounds can be active as agents that modulate the interaction of aldolases and transmembrane proteins in parasites such as, Cryptosporidium spp., Toxoplasma spp., Eimeria spp., and Babesia spp., for example.

As such, in accordance with another embodiment, the present invention provides the use of an agent that modulates the interaction between Cryptosporidium spp., Toxoplasma spp., Eimeria spp., and Babesia spp. aldolase and a transmembrane adhesive protein for use in treating a Cryptosporidium spp., Toxoplasma spp., Eimeria spp., and Babesia spp. infection in a subject.

Similarly, methods of inhibiting/killing Cryptosporidium spp., Toxoplasma spp., Eimeria spp., and Babesia spp. sporozoites are carried out by contacting a Cryptosporidium spp., Toxoplasma spp., Eimeria spp., and Babesia spp. sporozoite with an agent that modulates the interaction between Cryptosporidium spp., Toxoplasma spp., Eimeria spp., and Babesia spp. aldolase and a transmembrane adhesive protein. Methods of preventing proliferation of Cryptosporidium spp., Toxoplasma spp., Eimeria spp., and Babesia spp. sporozoites are also carried out by contacting a Cryptosporidium spp., Toxoplasma spp., Eimeria spp., and Babesia spp. sporozoite with an agent that modulates the interaction between Cryptosporidium spp., Toxoplasma spp., Eimeria spp., and Babesia spp. aldolase and a transmembrane adhesive protein.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

For example, the agent comprises a small molecule. Suitable small molecules included in the methods and uses disclosed herein comprise 4-{2-[(3,4-dimethylphenyl)amino]-1,3-thiazol-4-yl}-N,N-diethylbenzenesulfonamide hydrobromide (IUPAC name: 4-[2-(3,4-dimethylanilino)-1,3-thiazol-4-yl]-N,N-diethylbenzenesulfonamide; hydrobromide; Compound 3); N,N′-bis(4-bromophenyl)-2,3-quinoxalinediamine (IUPAC name: 2-N,3-N-bis(4-bromophenyl)quinoxaline-2,3-diamine; Compound 4); 3-bromo-N-[4-(1H-naphtho[2,3-d]imidazol-2-yl)phenyl]benzamide (IUPAC name: N-[4-(1H-benzo[f]benzimidazol-2-yl)phenyl]-3-bromobenzamide; Compound 17); 2-amino-N-benzyl-1-butyl-1H-pyrrolo[2,3-b]quinoxaline-3-carboxamide (IUPAC name: 2-amino-N-benzyl-1-butylpyrrolo[3,2-b]quinoxaline-3-carboxamide; Compound 18); ({1-[2-(4-methoxyphenyl)ethyl]-4-piperidinyl}methyl)methyl[(1-phenyl-1H-pyrazol-4-yl)methyl]amine (IUPAC name: 1-[1-[2-(4-methoxyphenyl)ethyl]piperidin-4-yl]-N-methyl-N-[(1-phenylpyrazol-4-yl)methyl]methanamine; Compound 23) and derivatives thereof.

It will be understood by those of ordinary skill in the art that in some embodiments, the methods can include use of one or more of the above agents in a single composition, or serially as administration of a first agent, followed by administration of a second, third, or fourth agent, either contemporaneously, or separated by time intervals of hours, days, weeks or months. These agents, individually or collectively, can also be combined with other antiparasitical or chemotherapeutic agents known or described herein.

Pharmaceutically acceptable salts are art-recognized, and include relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl) aminoethane; and the like, see, for example, J. Pharm. Sci., 66: 1-19 (1977).

In accordance with an embodiment, the present invention provides pharmaceutical compositions comprising the small molecules described above, and a pharmaceutically acceptable carrier.

In accordance with another embodiment, the present invention provides a composition comprising at least one or more of the small molecules selected from the group consisting of: 4-{2-[(3,4-dimethylphenyl)amino]-1,3-thiazol-4-yl}-N,N-diethylbenzenesulfonamide hydrobromide (IUPAC name: 4-[2-(3,4-dimethylanilino)-1,3-thiazol-4-yl]-N,N-diethylbenzenesulfonamide; hydrobromide; Compound 3); N,N′-bis(4-bromophenyl)-2,3-quinoxalinediamine (IUPAC name: 2-N,3-N-bis(4-bromophenyl)quinoxaline-2,3-diamine; Compound 4); 3-bromo-N-[4-(1H-naphtho[2,3-d]imidazol-2-yl)phenyl]benzamide (IUPAC name: N-[4-(1H-benzo[f]benzimidazol-2-yl)phenyl]-3-bromobenzamide; Compound 17); 2-amino-N-benzyl-1-butyl-1H-pyrrolo[2,3-b]quinoxaline-3-carboxamide (IUPAC name: 2-amino-N-benzyl-1-butylpyrrolo[3,2-b]quinoxaline-3-carboxamide; Compound 18); ({1-[2-(4-methoxyphenyl)ethyl]-4-piperidinyl}methyl)methyl[(1-phenyl-1H-pyrazol-4-yl)methyl]amine (IUPAC name: 1-[1-[2-(4-methoxyphenyl)ethyl]piperidin-4-yl]-N-methyl-N-[(1-phenylpyrazol-4-yl)methyl]methanamine; Compound 23) and derivatives thereof, and a pharmaceutically acceptable carrier.

Furthermore, in accordance with an embodiment, the present invention provides methods of inducing cell death/reducing viability, and/or inhibiting/killing sporozoites, and/or preventing proliferation of sporozoites in Plasmodium spp., Cryptosporidium spp., Toxoplasma spp., Eimeria spp., and Babesia spp. are carried out by contacting the sporozoite with a pharmaceutical composition described above, that modulates the interaction between Plasmodium spp., Cryptosporidium spp., Toxoplasma spp., Eimeria spp., and Babesia spp. aldolase and a transmembrane adhesive protein.

In accordance with another embodiment, the present invention provides the use of a pharmaceutical composition described above, that modulates the interaction between Plasmodium spp., Cryptosporidium spp., Toxoplasma spp., Eimeria spp., and Babesia spp. aldolase and a transmembrane adhesive protein for use in inducing cell death in a Plasmodium spp., Cryptosporidium spp., Toxoplasma spp., Eimeria spp., and Babesia spp.

The small molecule is administered at a concentration of 1 μM-2,500 μM, e.g., 10 μM, 25 μM, 50 μM, 75 μM, 100 μM, 150 μM, 175 μM, 200 μM, 250 μM, 300 μM, 350 μM, 400 μM, 500 μM, 600 μM, 700 μM, 800 μM, 900 μM, or 2,500 μM. For example, 2-amino-N-benzyl-1-butylpyrrolo[3,2-b]quinoxaline-3-carboxamide (Compound #18) is administered at EC₉₀˜30 μM. In another example, N,N′-bis(4-bromophenyl)-2,3-quinoxalinediamine (Compound 34) is administered at EC₉₀˜125 μM.

In some embodiments, the small molecules of the present invention are administered to a subject at a concentration of 1 μM-1,000 μM, including, for example, concentration ranges of 1 μM to 10 μM, 20 μM, 30 μM, 40 μM, 50 μM, 60 μM, 70 μM, 80 μM, 90 μM, 100 μM, 200 μM, 300 μM, 400 μM, 500 μM up to 1000 μM.

In accordance with another embodiment, the present invention provides methods utilizing the pharmaceutical compositions described above and at least one or more other antiparasitical compounds.

As used herein, the term “antiparasitical compound” means one or more active agents from the class of drugs known as antiprotozoals, anthelmintics, ectoparasiticides, and similar compounds. These groups include antiamebiasis agents, antifascioliasis agents, antifiliariasis agents, antileshmaniasis agents, antimalarials, antischistosomal agents, antitapeworm agents and antitrypanosomiasis agents. Examples of such compounds include ornithine, arsenicals, benzamidine, napthalenesulfonate, nitroimidazole, macrolides, nitrofuran, pentavalent anitmonials, phosphoryl choline, neomycin, thiazole, aminoacridine, oxyquinoline, tetracycline, trimethoprim/sulfamethoxazole, pyrimethamine, aminoquinolines, 4-methanolquinolines, biguanides, sulfonamides, sesquiterpene lactones, atovaquone, pyronaridine, piperaquine, artesunate-amodiaquine, nitroimidazole derivatives, Ivermectin, and related compounds. Also included in the term “antiparasitical compounds” are vaccines and antibodies to infectious parasites.

Embodiments of the invention also include a process for preparing pharmaceutical products comprising the compounds. The term “pharmaceutical product” means a composition suitable for pharmaceutical use (pharmaceutical composition), as defined herein. Pharmaceutical compositions formulated for particular applications comprising the compounds of the present invention are also part of this invention, and are to be considered an embodiment thereof.

As used herein, the term “treat,” as well as words stemming therefrom, includes preventative as well as disorder remitative treatment. The terms “reduce,” “suppress,” “prevent,” and “inhibit,” as well as words stemming therefrom, have their commonly understood meaning of lessening or decreasing. These words do not necessarily imply 100% or complete treatment, reduction, suppression, or inhibition.

With respect to pharmaceutical compositions described herein, the pharmaceutically acceptable carrier can be any of those conventionally used, and is limited only by physico-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. Examples of the pharmaceutically acceptable carriers include soluble carriers such as known buffers, which can be physiologically acceptable (e.g., phosphate buffer) as well as solid compositions such as solid-state carriers or latex beads. It is preferred that the pharmaceutically acceptable carrier be one which is chemically inert to the active agent(s), and one which has little or no detrimental side effects or toxicity under the conditions of use.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

In addition, in an embodiment, the compounds of the present invention may further comprise, for example, binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzalkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular compound, as well as by the particular method used to administer the compound. Accordingly, there are a variety of suitable formulations of the pharmaceutical composition of the invention. The following formulations for parenteral, subcutaneous, intravenous, intramuscular, intraarterial, intrathecal and interperitoneal administration are exemplary, and are in no way limiting. More than one route can be used to administer the compounds, and in certain instances, a particular route can provide a more immediate and more effective response than another route.

Suitable soaps for use in parenteral formulations include, for example, fatty alkali metal, ammonium, and triethanolamine salts, and suitable detergents include, for example, (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the compounds in solution. Preservatives and buffers may be used. In order to minimize or eliminate irritation at the site of injection, such compositions may contain one or more nonionic surfactants, for example, having a hydrophile-lipophile balance (HLB) of from about 12 to about 17. The quantity of surfactant in such formulations will typically range from about 5% to about 15% by weight. Suitable surfactants include, for example, polyethylene glycol sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

For purposes of the invention, the amount or dose of the compounds, salts, solvates, or stereoisomers of any one the compounds of Formula I, as set forth above, administered should be sufficient to effect, e.g., a therapeutic or prophylactic response, in the subject over a reasonable time frame. The dose will be determined by the efficacy of the particular compound and the condition of a human, as well as the body weight of a human to be treated.

The dose of the compounds, salts, solvates, or stereoisomers of any one the compounds of Formula I, as set forth above, of the present invention also will be determined by the existence, nature and extent of any adverse side effects that might accompany the administration of a particular compound. Typically, an attending physician will decide the dosage of the compound with which to treat each individual patient, taking into consideration a variety of factors, such as age, body weight, general health, diet, sex, compound to be administered, route of administration, and the severity of the condition being treated.

Alternatively, the compounds of the present invention can be modified into a depot form, such that the manner in which the compound is released into the body to which it is administered is controlled with respect to time and location within the body (see, for example, U.S. Pat. No. 4,450,150). Depot forms of compounds can be, for example, an implantable composition comprising the compound and a porous or non-porous material, such as a polymer, wherein the compound is encapsulated by or diffused throughout the material and/or degradation of the non-porous material. The depot is then implanted into the desired location within the body and the compounds are released from the implant at a predetermined rate.

In one embodiment, the compounds of the present invention provided herein can be controlled release compositions, i.e., compositions in which the one or more compounds are released over a period of time after administration. Controlled or sustained release compositions include formulation in lipophilic depots (e.g., fatty acids, waxes, oils). In another embodiment the composition is an immediate release composition, i.e., a composition in which all, or substantially all of the compound, is released immediately after administration.

In yet another embodiment, the compounds of the present invention can be delivered in a controlled release system. For example, the agent may be administered using intravenous infusion, an implantable osmotic pump, a transdermal patch, or other modes of administration. In an embodiment, a pump may be used. In one embodiment, polymeric materials can be used. In yet another embodiment, a controlled release system can be placed in proximity to the therapeutic target, i.e., the brain, thus requiring only a fraction of the systemic dose (see, e.g., Design of Controlled Release Drug Delivery Systems, Xiaoling Li and Bhaskara R. Jasti eds. (McGraw-Hill, 2006)).

The compounds included in the pharmaceutical compositions of the present invention may also include incorporation of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In some embodiments other solubilizing agents, such as cyclodextrins and derivatives thereof can be used in the formulation of the present invention.

In accordance with the present invention, the compounds of the present invention may be modified by, for example, the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection, than do the corresponding unmodified compounds. Such modifications may also increase the compounds' solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently, or in lower doses than with the unmodified compound.

The small molecule is administered once per month, twice per month, once per week, twice per week, once per day, twice per day, every 4 hours, or every hour for the duration required to treat or prevent malaria, e.g., at least one day, at least one week, at least two weeks, at least one month, at least three months, at least six months, at least nine months, or at least one year.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

Plasmodium species that cause malaria include Plasmodium vivax, Plasmodium ovale, Plasmodium malariae, Plasmodium falciparum, and Plasmodium knowlesi. As such, the methods and uses described herein are useful in treating malaria caused by these species of Plasmodium.

In some embodiments, the transmembrane adhesive protein comprises a thrombospondin-related anonymous protein (TRAP (sporozoite surface protein 2); PlasmoDB code for P. falciparum PF3D7_1335900, incorporated herein by reference), a merozoite-TRAP-like protein (MTRAP; PlasmoDB code for P. falciparum 3D7_1028700, incorporated herein by reference), or a circumsporozoite- and TRAP related protein (CTRP; PlasmoDB code for P. falciparum PF3D7_0315200, incorporated herein by reference). TRAP is utilized for liver cell invasion, MTRAP is utilized for blood cell invasion, and CTRP is needed during the skin traversal of the parasite as well as in the mosquito stage. The three proteins connect in a similar way with Aldolase.

Optionally, the interaction between Plasmodium aldolase and a transmembrane adhesive protein is stabilized. Alternatively, the interaction between Plasmodium aldolase and a transmembrane adhesive protein is inhibited.

In some cases, a composition of the invention is administered orally or systemically. Other modes of administration include rectal, topical (e.g., via a spray or a lotion), intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

A method of identifying a candidate compound that modulates the activity of Plasmodium spp., Cryptosporidium spp., Toxoplasma spp., Eimeria spp., and Babesia spp. aldolase is carried out by contacting Plasmodium spp., Cryptosporidium spp., Toxoplasma spp., Eimeria spp., and Babesia spp. aldolase with a candidate compound and determining whether the candidate compound modulates the interaction between Plasmodium spp., Cryptosporidium spp., Toxoplasma spp., Eimeria spp., and Babesia spp. aldolase and a transmembrane adhesive protein. Preferably, the candidate compound inhibits the interaction between Plasmodium spp., Cryptosporidium spp., Toxoplasma spp., Eimeria spp., and Babesia spp. aldolase and a transmembrane adhesive protein. Exemplary transmembrane adhesive proteins include a thrombospondin-related anonymous protein (TRAP), a merozoite-TRAP-like protein (MTRAP), or a circumsporozoite and TRAP related protein (CTRP).

By “binding to” a molecule is meant having a physicochemical affinity for that molecule.

“Detect” refers to identifying the presence, absence or amount of the analyte to be detected.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. Purity and homogeneity are typically determined using analytical chemistry techniques, for example, polyacrylamide gel electrophoresis or high performance liquid chromatography.

By “reduces” is meant a negative alteration of at least 5%, 10%, 25%, 50%, 75%, or 100%.

By “specifically binds” is meant a compound that recognizes and binds a polypeptide of the invention, but which does not substantially recognize and bind other molecules in a sample, for example, a biological sample, which naturally includes a polypeptide of the invention.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### EXAMPLES

### Example 1

Sporozoite Gliding Reduction.

Compounds selected from the initial hit list were tested using in vitro gliding assays. Five compounds were selected for dose dependent testing on gliding motility based on early crystallographic experiments and first round gliding assay experiments at high concentration.

Compounds.

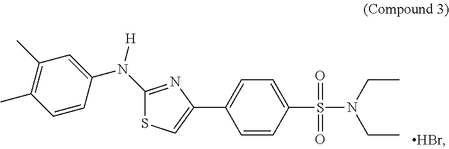 [SMILES: CCN(CC)S(=O)(=O)c1ccc(-c2csc(Nc3ccc(C)c(C)c3)n2)cc1]

4-{2-[(3,4-dimethylphenyl)amino]-1,3-thiazol-4-yl}-N,N-diethylbenzenesulfonamide hydrobromide (IUPAC name: 4-[2-(3,4-dimethylanilino)-1,3-thiazol-4-yl]-N,N-diethylbenzenesulfonamide; hydrobromide);

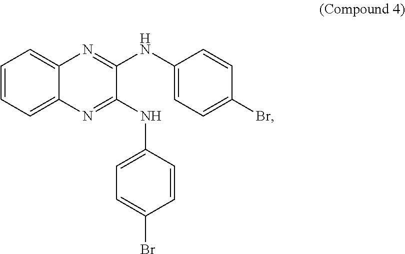 [SMILES: Brc1ccc(Nc2nc3ccccc3nc2Nc2ccc(Br)cc2)cc1]

N,N′-bis(4-bromophenyl)-2,3-quinoxalinediamine (IUPAC name: 2-N,3-N-bis(4-bromophenyl)quinoxaline-2,3-diamine);

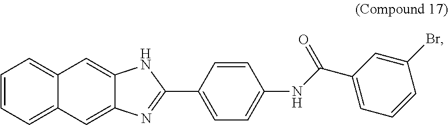 [SMILES: O=C(Nc1ccc(-c2nc3cc4ccccc4cc3[nH]2)cc1)c1cccc(Br)c1]

3-bromo-N-[4-(1H-naphtho[2,3-d]imidazol-2-yl)phenyl]benzamide (IUPAC name: N-[4-(1H-benzo[f]benzimidazol-2-yl)phenyl]-3-bromobenzamide);

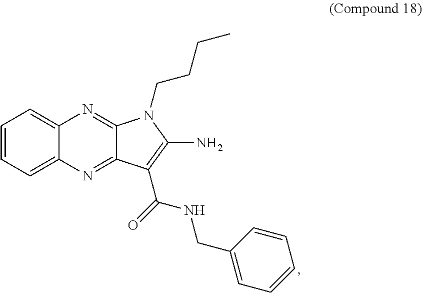 [SMILES: CCCCn1c(N)c(C(=O)NCc2ccccc2)c2nc3ccccc3nc21]

2-amino-N-benzyl-1-butyl-1H-pyrrolo[2,3-b]quinoxaline-3-carboxamide (IUPAC name: 2-amino-N-benzyl-1-butylpyrrolo[3,2-b]quinoxaline-3-carboxamide); and

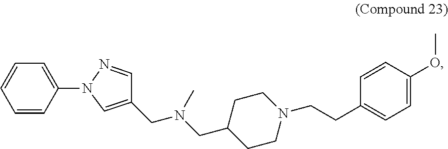 [SMILES: COc1ccc(CCN2CCC(CN(C)Cc3cnn(-c4ccccc4)c3)CC2)cc1]

({1-[2-(4-methoxyphenyl)ethyl]-4-piperidinyl}methyl)methyl[(1-phenyl-1H-pyrazol-4-yl)methyl]amine (IUPAC name: 1-[1-[2-(4-methoxyphenyl)ethyl]piperidin-4-yl]-N-methyl-N-[(1-phenylpyrazol-4-yl)methyl]methanamine) were selected for concentration-dependent gliding assays (Table 1).

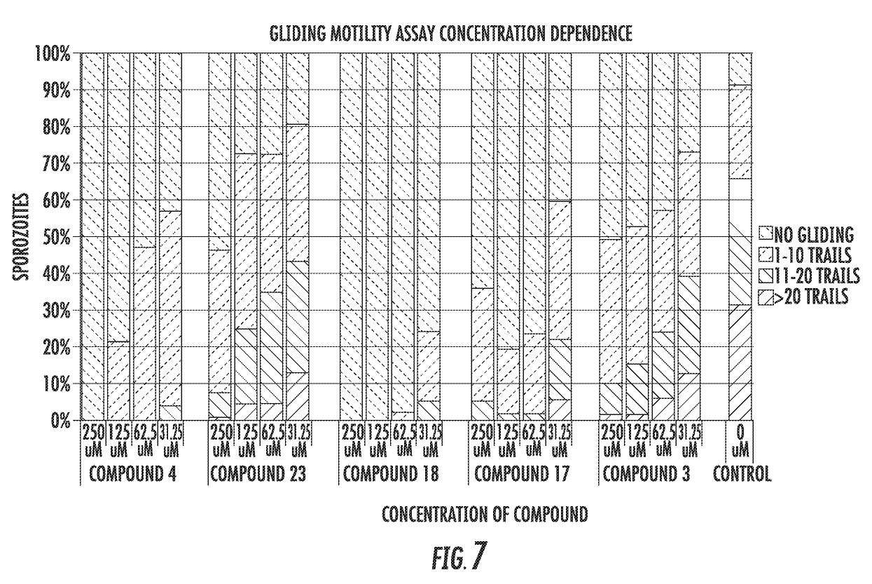

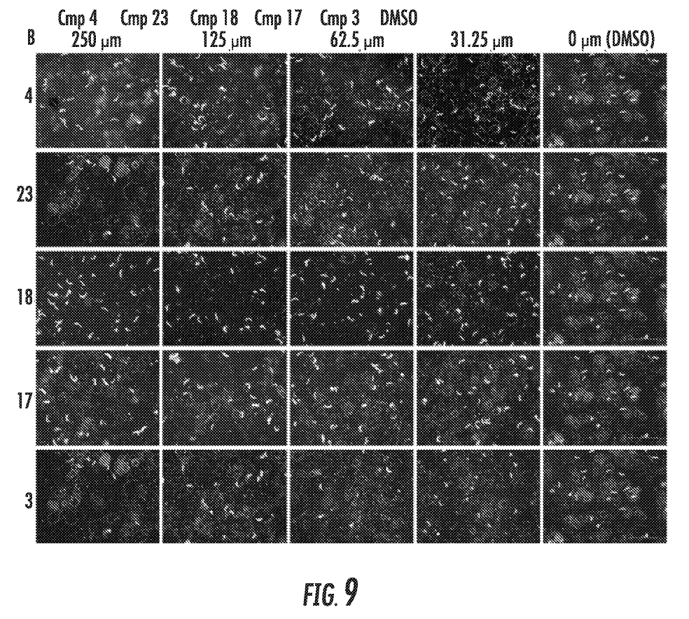

P. berghei sporozoites were purified from mosquitoes, incubated with compound, and allowed to glide on anti-CSP coated slides. Representative fields of views for sporozoites in the presence of each compound are shown in FIG. 9. The CSP-trails deposited by the gliding parasites were detected with anti-CSP antibodies. Trails were quantified for each sporozoite. The ability of each sporozoite to glide was categorized as no gliding (0 trails), and low (<10 trails), medium (10-20 trails), and high gliding (>20 trails). The quantified results for four different concentrations of each of the five compounds are shown in FIG. 7.

All compounds showed inhibition throughout the concentration range tested (2-fold dilutions, 250-31.25 μM). Compounds 4 and 18 had the greatest effect on gliding motility. In the DMSO control, more than 90% of sporozoites deposited trails, with >60% classified as medium and high gliding (>10 trails). In comparison, at 250 μM, compound 4 and compound 18 showed less than 10% total parasite gliding. At lower concentrations of compounds, the sporozoites did start to recover their gliding ability. However, for compound 4 at the lowest concentration of compound, less than 60% of the sporozoites were gliding and when they did glide, 90% of those sporozoites were depositing less than 10 trails and upon inspection of multiple fields, they typically could only complete one circular trail. For compound 18, less than 25% of sporozoites were able to glide at the lowest compound concentration and 75% of those sporozoites were low gliding, with less than 10 trails deposited; however, these low gliding parasites typically deposited more than 1 trail, unlike those from compound 4. These results indicate that gliding motility is affected in the presence of the compounds. However, from this single assay, it is not possible to assess whether the compounds were directly affecting the motor through the proposed TRAP-Aldolase interaction, by affecting an energy production step necessary to generate ATP for motor power, or by cytocidal effect.

TABLE 1
Table of compounds pursued for in vitro parasite testing.

| | ChEMBL | Chembridge | |
|---|---|---|---|
| Compound | ID | ID | MMV ID |
| 3 | 604389 | 5251155 | 009108 |
| 4 | 537778 | 5317392 | 007224 |
| 17 | 587485 | 7078022 | 019241 |
| 18 | n/a | 7881935 | 396744 |
| | | | (analog) |
| 23 | 533399 | 67378588 | 666069 |

### Example 2

Altered Gliding Phenotypes.

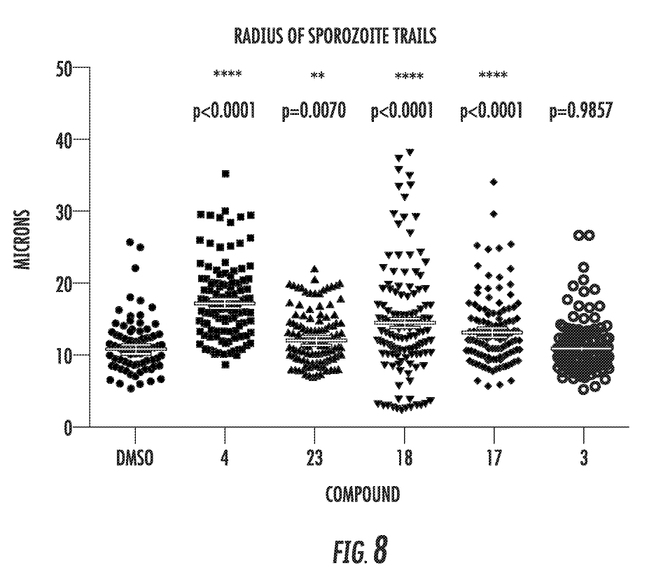

The gliding phenotype was altered to determine whether the compounds were having off-target effects. While quantifying the number of deposited trails, the trail diameter in the presence of compounds was altered. An altered trail diameter is more likely due to either a direct effect on the motor or an effect on sporozoite shape. The diameter of the circular trails deposited by the parasites in the DMSO control was measured and in the presence of three compounds at 62.5 μM (FIG. 8). This concentration was chosen because at higher concentrations, there were not enough sporozoite trails to measure to do a statistical analysis of the results. Using ImageJ, circles were fit to trails in multiple images to measure 130-150 trail radii. The average trail radii increased in the presence of compounds 4, 17, and 18 with a p-value <0.0001. The increase in average trail size was 6.3 microns, 2.5 microns, and 3.6, for compounds 4, 17, and 18, respectively. This result showed that an off target effect was not observed.

### Example 3

Sporozoite-Gliding.

To determine the compound effects on the speed of gliding and type of gliding movement, live imaging of mCherry expressing parasites was used. P. berghei parasites constitutively expressing mCherry were allowed to settle on slides and imaged using an inverted microscope at 1.57 fps. ImageJ and the ToAST plugin were used to track parasites for a minimum of 150 frames in the 200 frame movies. The coordinates obtained via the automated tracking were much more useful than those using manual tracking. The ToAST plugin provides automatic identification of sporozoites from debris by aspect ratio and pixel analysis. The program also measures velocity and determine whether sporozoites are gliding in counterclockwise (CCW) or clockwise (CW) patterns, or whether they are stationary or waving, with large changes in vector angles between images. However, the automatic assignment of gliding pattern resulted in error and could not accept non-integer fps values.

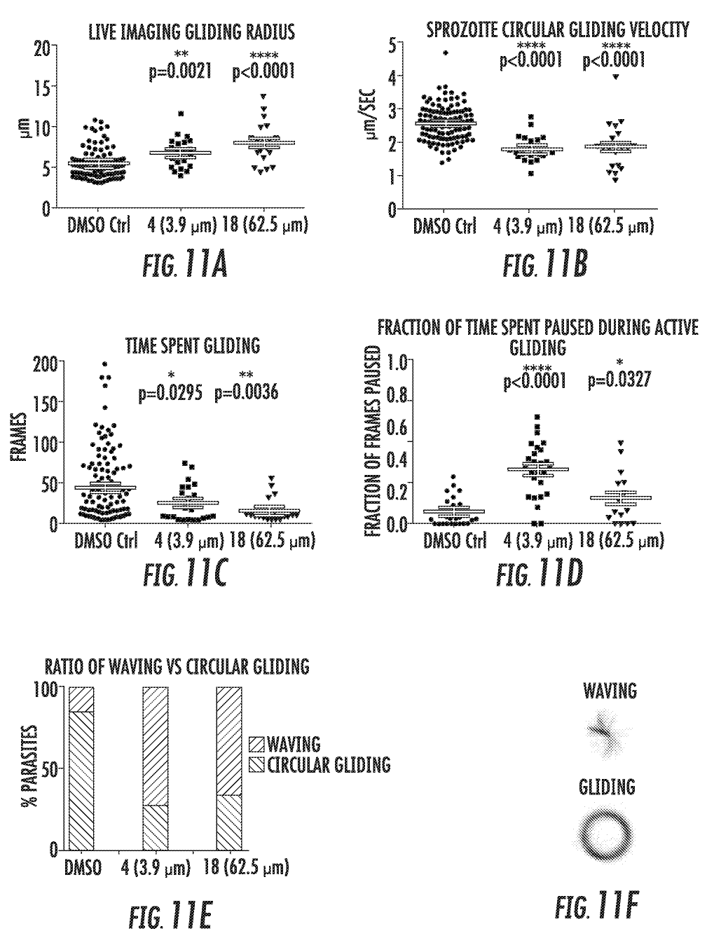

Instead, the output position data was reanalyzed using similar algorithms as defined by ToAST. Sporozoites were assigned to one of four states at each time point, CW gliding, CCW gliding, stationary, or waving. To determine the speed of gliding, the average velocity was determined for sections of time in which the sporozoite was gliding continuously for more than 6 frames. Sporozoites would often glide for a number of frames and then stop gliding for a number of frames before continuing movement, resulting in more than one block of frames representing continuous gliding. The average for each sporozoite was a weighted average of the average speed during each block of gliding. The DMSO control parasites traveled in a CCW and CW pattern at an average speed of 2.58+/−0.05 um/s. Compounds 4 and 18 showed a significant reduction (p-value <0.0001) in sporozoite gliding (FIG. 11B). At 62.5 μM, compound 18 had an average gliding velocity of 1.88+/−0.17 um/s. At 3.9 μM, compound 4 displayed an average gliding velocity of 1.81+/−0.09 um/s. Compound 4 gliding information was collected at a lower concentration compared to compound 18 because it was not possible to obtain a sufficient number of circularly gliding parasites for analysis at the higher concentrations (FIG. 11A).

As previously mentioned, sporozoites would often glide for several frames, then stop or pause, and then continue gliding. Upon initial observation of compound 4 samples, it was observed that parasites tended to glide in a stop-and-go manner, gliding for 4-6 frames and then stopping for 2-3 frames. To determine whether these pauses deviated from those observed in the control, the movies were analyzed to determine the total amount of time spent circularly gliding over the course of the 200 frame movies (FIG. 11C). Additionally, the fraction of time that the sporozoites paused for during active gliding was measured (FIG. 11D). A pause was defined as a cessation of circular gliding for no more than 10 frames. The fraction of paused frames to moving frames was measured for stretches of active gliding. The total time spent gliding was significantly less for both compound 4 and 18, at 25 and 16 frames, respectively, compared to the control at 46 frames (FIG. 11C). For fraction of time spent paused, both compounds 4 and 18 deviated significantly from the DMSO control, which showed pauses for only 7% of active gliding time. Compound 4 increased pausing to 32% of the active gliding time, while compound 18 increased pauses to just 16% (FIG. 11D). The increase in pauses by compound 4 reflects the stop-and-go circular gliding that was observed for the sporozoites.

As automatic sporozoite classification using ToAST did not work properly with the acquired data, manual assignment of sporozoite motion was assigned from looking at z-stacks. Z-stack images were created using ImageJ using the average intensity in each frame. From these frames, patterns of movements to one of two categories were assigned, circular gliding or waving. Waving sporozoites are defined as having on end attached while the other end moves or waves freely (FIG. 11F), resulting in large angular changes in the direction of the vector that lies along the sporozoite. Examples of these movement behaviors as they appear in z-stacks are shown in FIG. 11E. Both compound 4 and 18 changed the ratio between gliding and waving sporozoites. In the control, nearly 85% of sporozoites are gliding circularly and 15% waving, whereas 72% and 66% are waving in compound 4 and 18 samples, respectively.

### Example 4

Sporozoite Viability Assays.

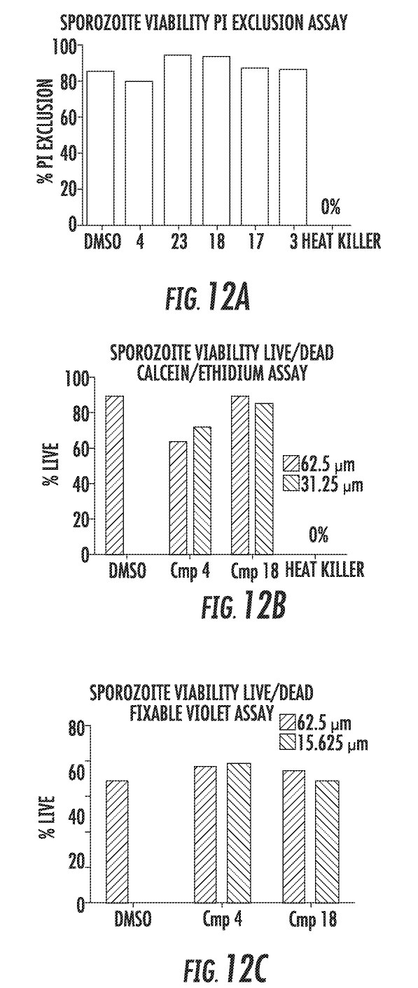

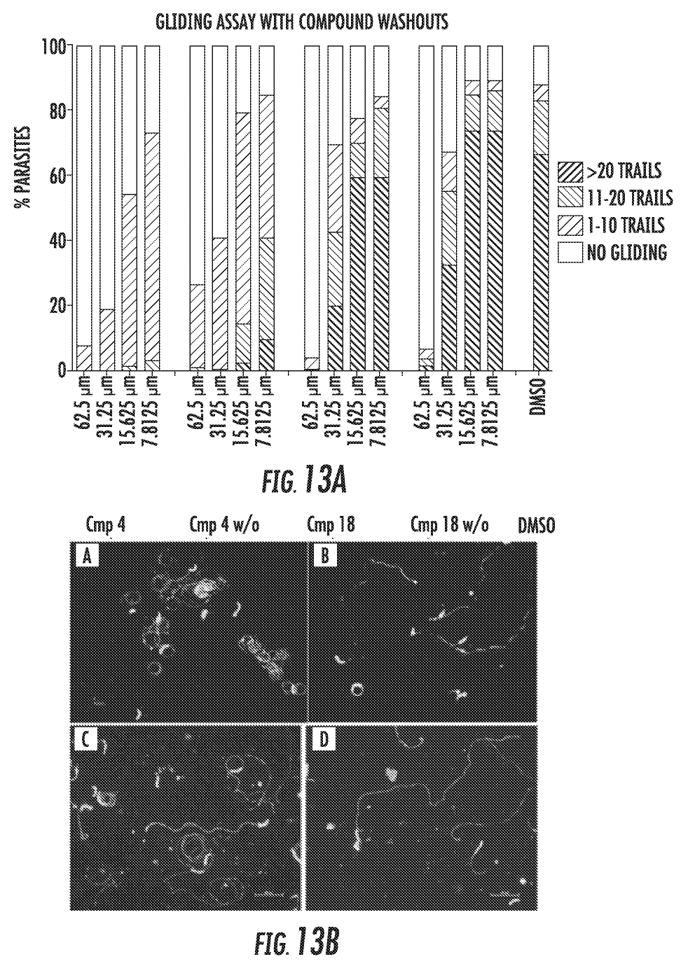

To determine if the compounds' mode of action was by causing cell death, viability assays to look at compound effects were performed. In a first test of viability, a propidium iodide exclusion assay was used (FIG. 12A). The total number of sporozoites was determined from in phase imaging and the number of non-viable sporozoites from the count of red fluorescing sporozoites. The total viability of sporozoites was determined as the percentage of sporozoites that did not fluoresce red in the presence of PI, indicating that the parasite was viable and could actively exclude the DNA binding dye. A 10 min incubation at 18° C. followed by 15 min at 37° C. in the presence of DMSO indicated that 85% of sporozoites were viable. 250 μM of compound 4 reduced viability to 79% while compound 18 indicated a protective effect with 93% viability.

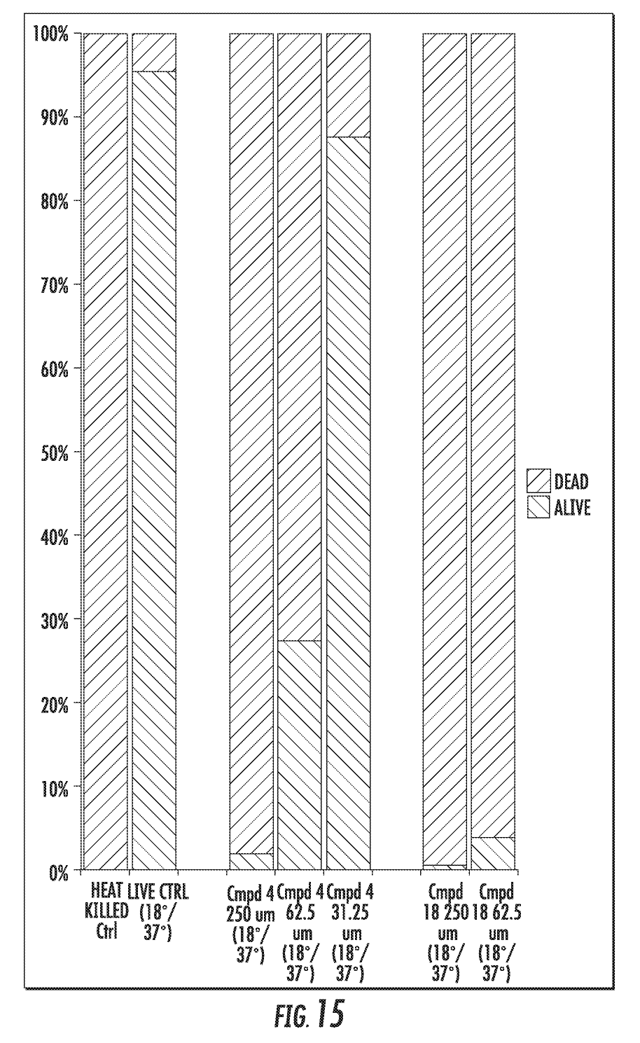

To further investigate viability, a 2 dye system of ethidium and calcein-AM was used to measure both membrane permeability, a marker of death, and enzyme activity, a marker of viability (FIG. 12B). Live cells with intact membranes are impermeable to the ethidium DNA dye but are permeable to the calcein-AM dye, which can interact with active esterases and will fluoresce green. Dying cells allow the ethidium to cross the membrane and interact with DNA, fluorescing red. This two color readout allowed discrimination between live versus dead sporozoites. Sporozoites were incubated at room temperature with two different concentrations of compounds for 15 min and then counted. Counting of the DMSO treated sporozoites showed that 96% of the cells were alive (green). Compound 4 and 18 at 62.5 μM reduced sporozoite viability to 82% and 88%, respectively. At these concentrations, assays could be performed such that the compounds would not kill the parasite. Instead, the compounds merely impaired parasite mobility, indicating an on-target effect of the small molecule on Aldolase-TRAP. By modulating this interaction, despite not killing the parasites at that point, a dramatic reduction in liver cell invasion was observed (FIG. 14A). An extended 15 min incubation at 37° C. had little effect on sporozoite viability, while compound 4 decreased sporozoite viability to 66% and compound 18 did not differ from the control. As these dyes are unable to be fixed and sporozoites are not viable for long periods of time, it is possible that there are some viability variances due to difference in time from incubation with the dye until counting.

The LIVE/DEAD assay indicated a greater decrease in viability in the presence of compound 4 and that compound 18 is relatively mild (FIG. 12C). However, the viability decrease is not high enough to account for the levels of non-gliding sporozoites observed in the gliding assays.

### Example 5

Gliding Assay Reversibility Testing with Compound Washouts.

A gliding motility assay with a compound washout step was also performed to measure the reversibility of the compound's effects. The sporozoites were incubated at for a total of 20 min with the compound before they were washed and re-suspend and allowed to glide. Compound 18 was completely reversible at lower concentrations of compound (e.g., 15.625 and 7.8125 μM). At higher concentrations of 31.25 μM, the number of high-gliding parasites increased when the compound was washed away, but the total number of sporozoites did not change much. At 62.5 μM, there was little reversibility observed. For compound 4, which seemed to be more detrimental to the sporozoite as determined by PI exclusion and LIVE/DEAD assays, no complete reversibility in washouts at any concentration was observed. When concentration of compound 4 was dropped to less than 8 μM, the total number of parasites gliding was comparable to wild type, however, the number of high gliding parasites was 10% of that observed for the DMSO control. These results indicate that compound 18 effects are reversible at lower concentrations while compound 4 effects are not reversible. Interestingly, compound 18 also showed higher levels of gliding in the non-washout control as compared to the first round of gliding assays performed. The control sporozoites also showed higher levels of gliding. This indicates that the sporozoite preparation yielded more viable sporozoites and that there may be compound batch variability.

### Example 6

Aldolase-TRAP Specific Interaction Measurement Via Surface Plasmon Resonance (SPR).

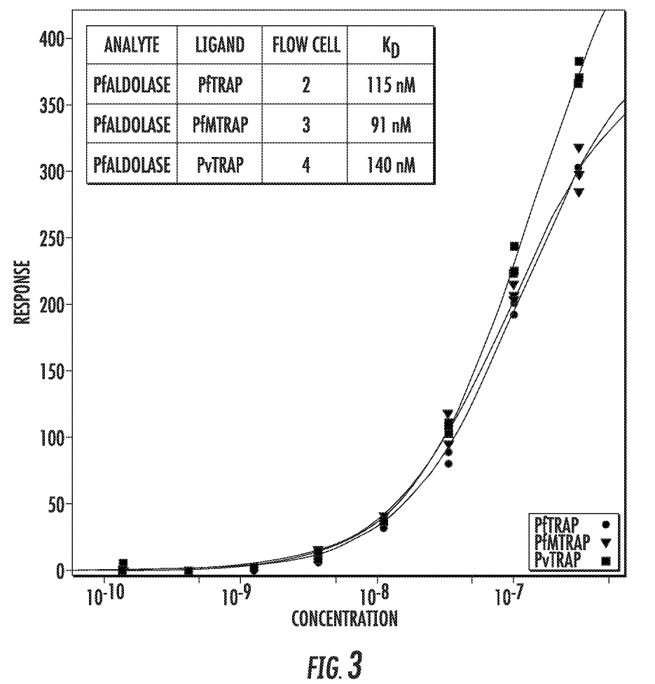

The dissociation constant for PfAldolase and PfTRAP, MTRAP and PvTRAP was measured by determining the binding response at various concentrations of aldolase passed over a chip with bound, biotinylated peptides (FIG. 3). Dissociation constants were in the mid-nanomolar range and matched with values previously reported in the literature (Boucher LE and Bosch J, J. Mol Recognit. 2013 Oct. 26(10): 496-500).

### Example 7

Screening of MMV Library for Compounds that Promote the TRAP-Aldolase Interaction.

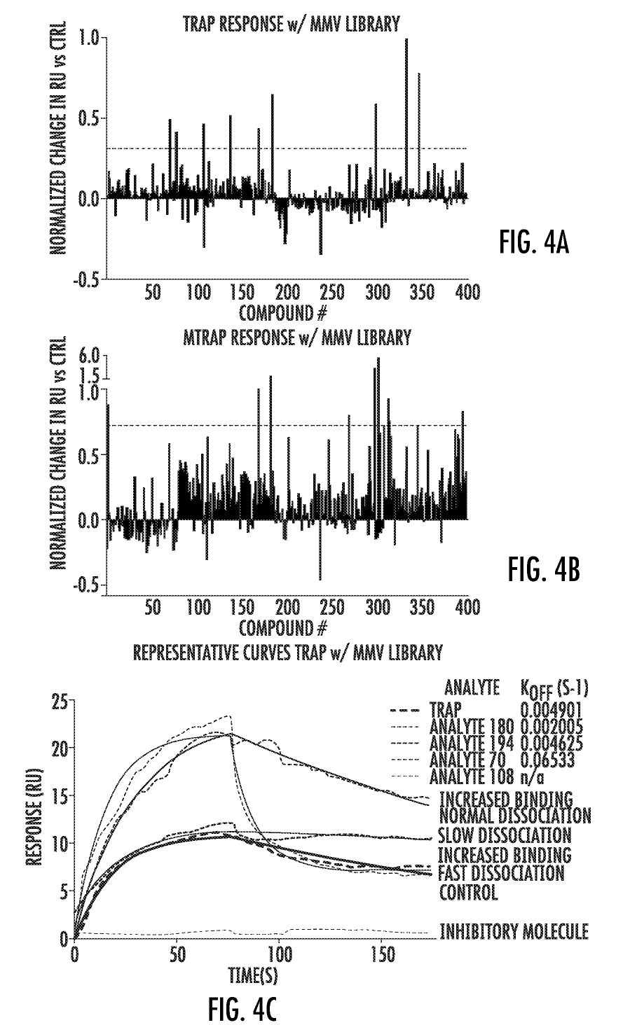

Response in the presence of compound is shown in graphs, normalized to the control (DMSO), which was set to zero. Positive responses indicated increased interaction between TRAP (FIG. 4A) or MTRAP, and aldolase and negative response indicated a disruption of the interaction (FIG. 4B). FIG. 4C shows selected binding curves from the screen. The control curve is shown in black. Examples of slow dissociation with normal binding (blue), increased binding with normal dissociation (green), increased binding with fast dissociation (pink) and disruption of the interaction (purple) are shown.

### Example 8

Increased Binding Interaction of (M)TRAP with Aldolase in the Presence of Compound.

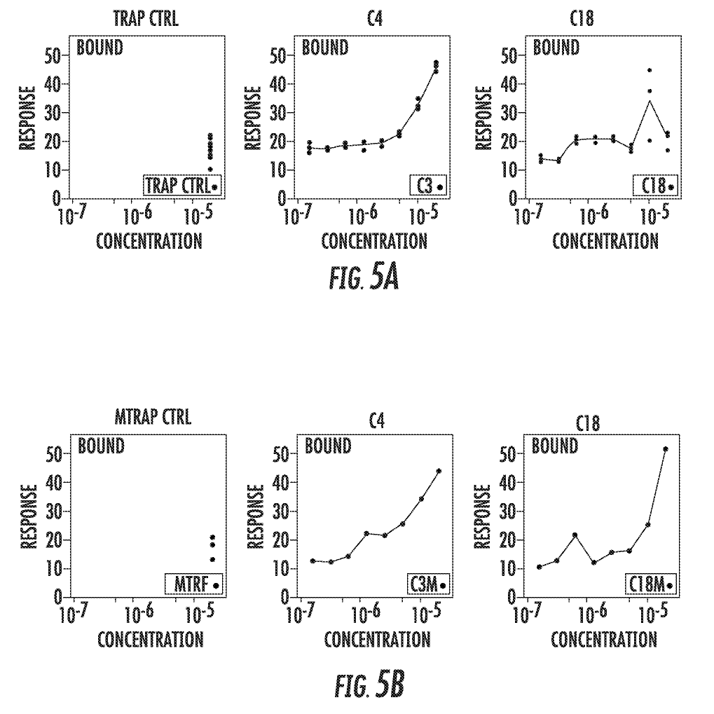

Increased binding interaction of (M)TRAP with aldolase in the presence of compound as a function of concentration was measured. (FIG. 5A) TRAP and (FIG. 5B) MTRAP interaction with aldolase in the presence of compounds 4 and 18 is illustrated as a function of compound concentration as measured via SPR.

### Example 9

Inhibition of Sporozoite Gliding Motility in the Presence of Compound.

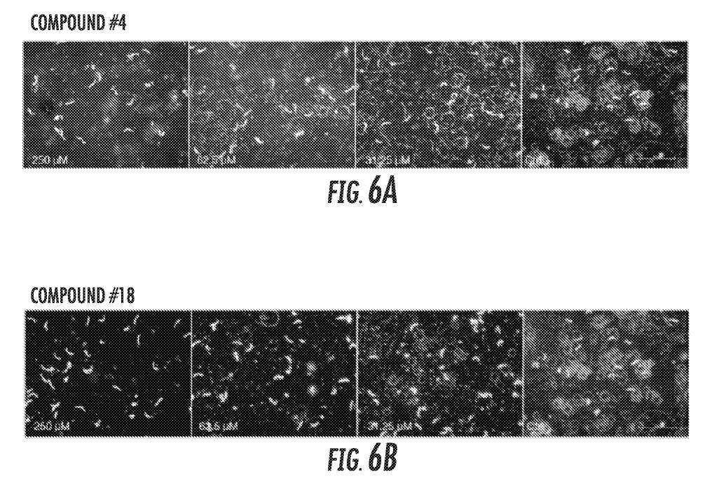

FIG. 6 shows inhibition of gliding motility in the presence of compound. Images of parasite CSP trails shed during gilding on antibody coated slides in the presence of decreasing concentrations of compound. Compound 4 (FIG. 6A) and compound 18 (FIG. 6B) effects on gliding are shown in comparison to DMSO control (right panels).

### Example 10

Views of Sporozoite in the Presence of Compounds.

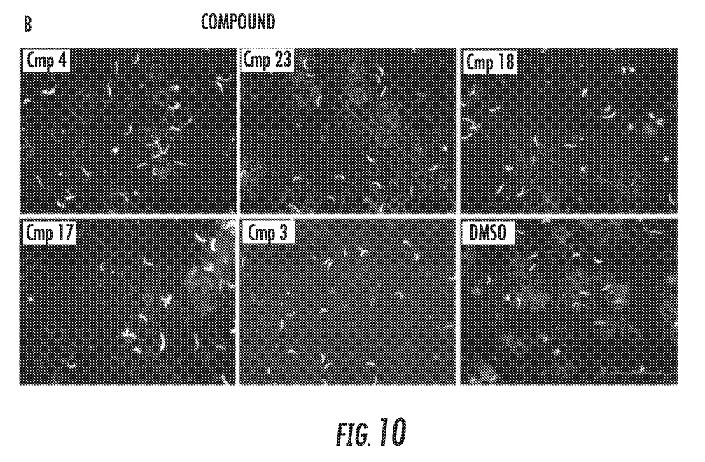

FIG. 10 shows representative views of sporozoite in the presence of compounds and DMSO control.

### Example 11

Gliding Assays with Compound Washouts.

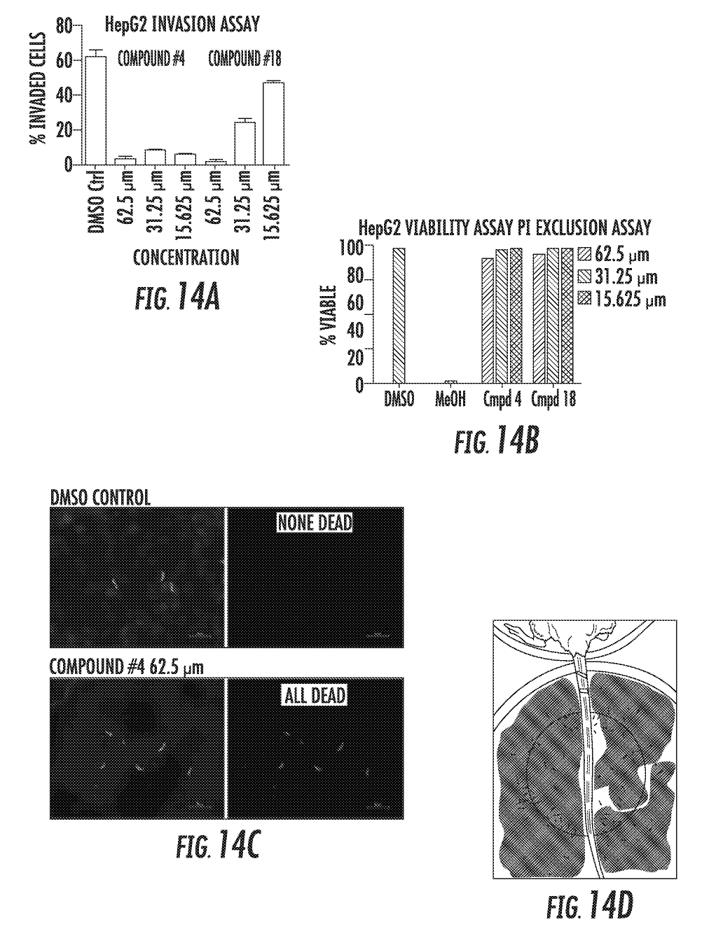

FIG. 13A shows gliding assays results with compound washouts, indicating partial recovery of motility when the compound is removed. FIG. 13B shows representative images from slides of gliding sporozoites. The top panels show images reprinted with permission from: American Chemical Society. Kortagere, S. et al. (2010) “Structure-based design of novel small-molecule inhibitors of Plasmodium falciparum.” J. Chem. Inf. Model, 50(5):840-9 [73]. DMSO treated sporozoites (a, upper left) with circular gliding pattern versus (b, upper right) pyrazole-urea compound treated sporozoites with a unique wavy line gliding pattern. Below are images of a compound 4 washout slide at 7.8 μM showing a similar wavy gliding patterns in addition to circular gliding (c,d).

### Example 12

HepG2 Invasion Assay in the Absence and Presence of Compounds.

FIG. 14 A shows histogram of HepG2 invasion assay results in the presence and absence of compounds. FIG. 14B shows HepG2 viability using propidium iodine exclusion assays in the absence and presence of compounds. FIG. 14C shows LIVE/DEAD assay of compound #4 at 62.41M compared to DMSO control. FIG. 14 D shows mouse tail used for testing of compound #4 and compound #18 as a topical agent. Compounds were applied onto the mouse tail solubilized in 100% DMSO at a concentration of 2.5 mM (˜100 fold of EC₉₀). Infected Anopheles stephensi mosquitoes carrying the P. berghei rodent malaria parasite were allowed to feed on the treated tails.

### Example 13

Compounds Kill Sporozoites.

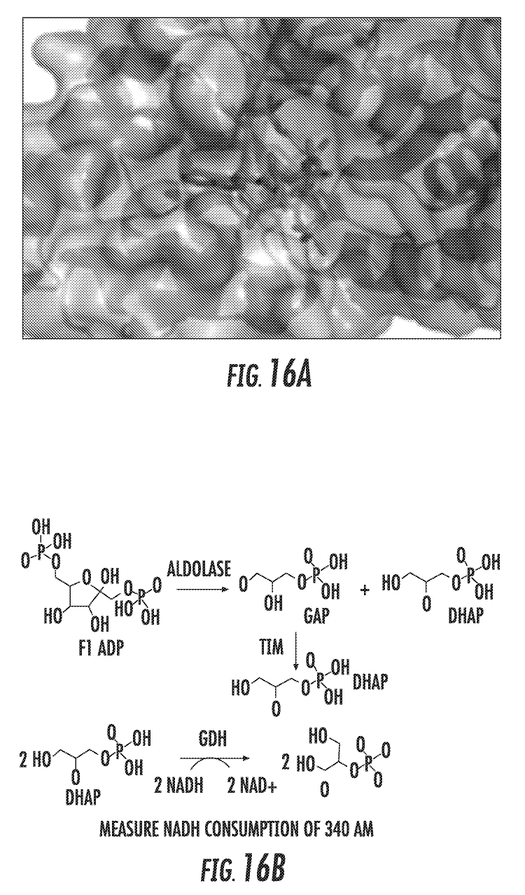

Dose-dependent killing of P. falciparum sporozoites is shown in FIG. 15. Compound 18 EC₉₀˜30 μM and compound 4 EC₉₀˜125 μM. Observed phenotype at lower concentration pinpoints towards an on-target effect of the compounds.

### Example 14

Dose-Dependent Enzymatic Inhibition of Compounds on P. falciparum Aldolase and Rabbit Aldolase as a Surrogate for Human Aldolase.

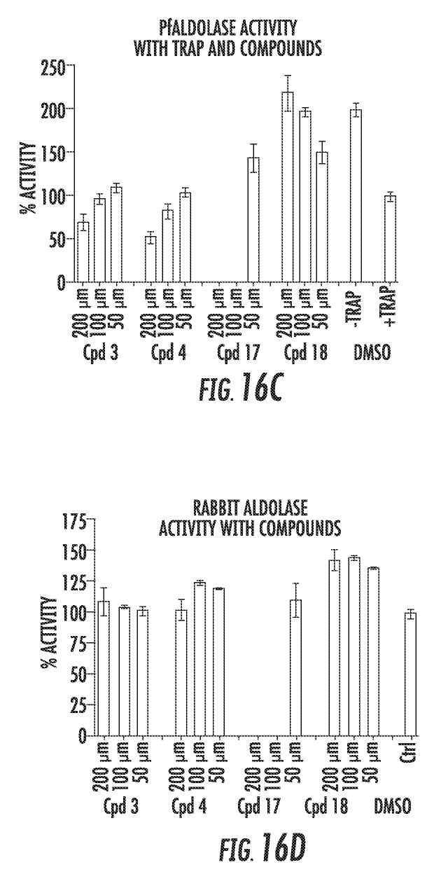

As shown in FIG. 16, compound 3 and 4 show no enzyme inhibition on rabbit aldolase while P. falciparum aldolase is inhibited in the presence of TRAP and the compounds, further evidence of the exquisite specificity of the small molecules of the present invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-18. (canceled)

19. A method of identifying a candidate compound that modulates the activity of Plasmodium spp., Cryptosporidium spp., Toxoplasma spp., Eimeria spp., and Babesia spp. aldolase comprising contacting Plasmodium spp., Cryptosporidium spp., Toxoplasma spp., Eimeria spp., and Babesia spp. aldolase with a candidate compound and determining whether said candidate compound modulates the interaction between Plasmodium spp., Cryptosporidium spp., Toxoplasma spp., Eimeria spp., and Babesia spp. aldolase and a transmembrane adhesive protein.

20. The method of claim 19, wherein said candidate compound inhibits the interaction between Plasmodium spp., Cryptosporidium spp., Toxoplasma spp., Eimeria spp., and Babesia spp. aldolase and a transmembrane adhesive protein.

21. The method of claim 20, wherein said transmembrane adhesive protein comprises a thrombospondin-related anonymous protein (TRAP), a merozoite-TRAP-like protein (MTRAP), or a circumsporozoite- and TRAP related protein (CTRP).

22. A method for treating a subject having a parasitic infection comprising administering to the subject an agent that modulates the interaction between aldolase and a transmembrane adhesive protein in the parasitic species.

23. The method of claim 22, wherein the infection is caused by one or more of the following species selected from the group consisting of Plasmodium spp., Cryptosporidium spp., Toxoplasma spp., Eimeria spp., and Babesia spp.

24. The method of claim 22, wherein the agent is a small molecule selected from the group consisting of: 4-{2-[(3,4-dimethylphenyl)amino]-1,3-thiazol-4-yl}-N,N-diethylbenzenesulfonamide hydrobromide (IUPAC name: 4-[2-(3,4-dimethylanilino)-1,3-thiazol-4-yl]-N,N-diethylbenzenesulfonamide; hydrobromide; Compound 3); N,N′-bis(4-bromophenyl)-2,3-quinoxalinediamine (IUPAC name: 2-N,3-N-bis(4-bromophenyl)quinoxaline-2,3-diamine; Compound 4); 3-bromo-N-[4-(1H-naphtho[2,3-d]imidazol-2-yl)phenyl]benzamide (IUPAC name: N-[4-(1H-benzo[f]benzimidazol-2-yl)phenyl]-3-bromobenzamide; Compound 17); 2-amino-N-benzyl-1-butyl-1H-pyrrolo[2,3-b]quinoxaline-3-carboxamide (IUPAC name: 2-amino-N-benzyl-1-butylpyrrolo[3,2-b]quinoxaline-3-carboxamide; Compound 18); and ({1-[2-(4-methoxyphenyl)ethyl]-4-piperidinyl}methyl)methyl[(1-phenyl-1H-pyrazol-4-yl)methyl]amine (IUPAC name: 1-[1-[2-(4-methoxyphenyl)ethyl]piperidin-4-yl]-N-methyl-N-[(1-phenylpyrazol-4-yl)methyl]methanamine; Compound 23).

25. The method of claim 24, wherein the use further comprises one or more additional antiparasitical agents.

26. The method of claim 24, wherein said small molecule is administered at a concentration of 1 μM-2,500 μM.

27. The method of claim 23, wherein said Plasmodium spp. comprises Plasmodium vivax, Plasmodium ovale, Plasmodium malariae, Plasmodium falciparum, or Plasmodium knowlesi.

28. The method of claim 22, wherein said transmembrane adhesive protein comprises a thrombospondin-related anonymous protein (TRAP), a merozoite-TRAP-like protein (MTRAP), or a circumsporozoite- and TRAP related protein (CTRP).

29. The method of claim 23, wherein said interaction between Plasmodium spp., Cryptosporidium spp., Toxoplasma spp., Eimeria spp., and Babesia spp. aldolase and a transmembrane adhesive protein is stabilized.

30. The method of claim 23, wherein said interaction between Plasmodium spp., Cryptosporidium spp., Toxoplasma spp., Eimeria spp., and Babesia spp. aldolase and a transmembrane adhesive protein is inhibited.

31. The method of claim 22, wherein the agent is combined with a pharmaceutically acceptable carrier.

32. The method of claim 31, wherein said agent is administered orally, topically, or intravenously.

33. The method of claim 31, wherein said agent is in the form of a spray or a lotion.
